(19) United States
(12) Patent Application Publication
(10) Pub. No.: US 2005/0053630 A1
(43) Pub. Date: Mar. 10, 2005

(54) Antiseptic/antifungal agent and endermic liniment composition which contains it

(76) Inventors: Shigeyuki Ogawa, Yokohama-Shi (JP); Yoshio Asaka, Yokohama-Shi (JP)

Correspondence Address: LAW OFFICES OF TOWNSEND & BANTA;South Building, Suite 900, 601 Pennsylvania Ave., N.W., Washington, DC, 20004, US

(21) Appl. No.: 10/623,174

(22) Filed: Jul. 21, 2003

Related U.S. Application Data
(63) Continuation-in-part of application No. 09/537,261, filed on Mar. 29, 2000, now Pat. No. 6,620,418.

(30) Foreign Application Priority Data
Mar. 30, 1999 (JP) 11-087741
Mar. 30, 1999 (JP) 11-089072
Jan. 18, 2000 (JP) 2000-0087461

(51) Int. Cl. A61K 7/00

(52) U.S. Cl. 424/401

(57) ABSTRACT

A method of preserving a cosmetic is provided, comprising mixing into the cosmetic a substantially paraben-free endermic liniment composition comprising, an antiseptic/antifungal agent for inhibition of microbial growth in cosmetics. The antiseptic/antifungal agent is comprised of 0.1-3 wt % 2-n-butyl-2-ethyl-1,3-propanediol, 2.0-5.0 wt % 1,3-butylene glycol, and water. Alternatively, the antiseptic/antifungal agent may comprise 0.1-3.0 wt % 2,2-diallyl-1,3-propanediol and 0.1-15.0 wt % of a diol. In addition, a substantially paraben-free endermic liniment composition comprising an antiseptic/antifungal agent for inhibition of microbial growth in cosmetics is provided, the agent comprising 0.1-3 wt % of 2-n-butyl-2-ethyl-1,3-propanediol, 2.0-5.0 wt % 1,3-butylene glycol, and water.

### RELATED APPLICATIONS

This application is a Continuation-In-Part application claiming the priority of parent U.S. patent application Ser. No. 09/537,261, filed Mar. 29, 2000, currently pending and allowed, which claims the priority of Japanese Patent application Nos. 11-087741 and No.11-089072, both filed on Mar. 30, 1999, and No.2000-008746 filed on Jan. 18, 2000, which are incorporated herein by reference.

### FIELD OF THE INVENTION

This invention relates to a antiseptic/antifungal agent and an endermic liniment composition which contains it.

### BACKGROUND OF THE INVENTION

Generally, an endermic liniment composition such as a cosmetic contains an antiseptic/antifungal agent (in the present specifications, “antiseptic” also means “antifungal” unless specified otherwise) such as paraoxy benzoate (so called parabens), salicylic acid, and sorbic acid and/or an antiseptic assistant such as phenoxy ethanol, for the purpose of improving the shelf life of the products by suppressing replication of microorganisms which contaminate the composition from outside and eventually killing off these in microorganisms.

Antiseptic components such as paraben and/or antiseptic assistant components such as phenoxy ethanol have superior safety and efficacy when used as an antiseptic component in an endermic liniment composition. However, they still may cause irritation and such to a small number of very sensitive users.

Recently, endermic liniment compositions which are gentler to the skin are more in demand, and therefore the requirements of today's endermic liniment compositions are very difficult to meet by simply adding these parabens and phenoxy ethanol as the antiseptic components.

Of course it is possible to create an endermic liniment composition which does not have antiseptic components such as parabens or antiseptic assistant components such as phenoxy ethanol. However, in such a case, in order to ensure the antiseptic properties, the amount and/or the expiration date have to be limited or a complex means such as small subdivided containers or the backless mechanism has to be used, resulting in a tendency towards low economic benefits and versatility.

### SUMMARY OF THE INVENTION

Therefore, the object of the present invention is to provide a new antiseptic agent which has much superior safety and usability and can be blended into endermic liniment compositions, as well as an endermic liniment composition which contains it.

The inventors conducted earnest research to achieve the aforementioned object, and, as a result, discovered that 2,2-dialkyl-1,3-propanediol has a superior antiseptic effect and that it can be used as the effective ingredient of an antiseptic/antifungal agent. The inventors further discovered that an endermic liniment composition containing this antiseptic/antifungal agent has a superior antiseptic effect and that, even when the amount of parabens and/or phenoxy ethanol is substantially reduced, an antiseptic properties adequate for normal use can be ensured. The inventors also discovered that, depending on the blend ratio of 2,2-dialkyl-1,3-propanediol, adequate antiseptic properties can be ensured in an endermic liniment composition without adding any parabens and/or phenoxy ethanol, thus completing the present invention.

That is, the present invention provides an antiseptic/antifungal agent in which 2,2-dialkyl-1,3-propanediol represented by the following general formula (I) is an effective component:

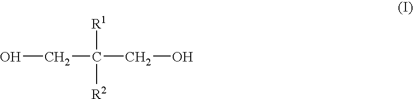

wherein, R¹ and R² can either be identical or different from each other, and both denote an alkyl group with a carbon number of 1-4.

The present invention further provides an endermic liniment composition which contains the antiseptic agent of the present invention (hereinafter referred to as “the endermic liniment composition of the present invention”).

As mentioned above, in the endermic liniment composition of the present invention, it is possible to use 2,2-dialkyl-1,3-propanediol represented by general formula (I) as essentially the only antiseptic agent.

The inventors conducted the aforementioned research, and, as a result, discovered that, by blending a combination of 3-methyl-3-methoxybutanol, which has been widely used as a solvent for perfumes, and 2-phenoxy ethanol, which has been widely used as an antiseptic assistant, or 1,2-pentanediol, which has been widely used as a humectant, a surprisingly superior anti septic effect is manifested and that antiseptic properties adequate for normal use can be ensured even if the blend ratios of parabens and/or phenoxy ethanol are significantly reduced. That is, although there have been examples of use of both of these compounds, they have not been used together, and the present invention discovered that the combined use of these compounds manifests a superior antiseptic effect and ensures the antiseptic properties.

The inventors discovered, in particular, that depending on the blend ratios when combining 3-methyl-3-methoxybutanol and 1,2-pentanediol, ad equate antiseptic properties can be ensured without using any parabens, and verified that usability and safety were excellent, thus completing the present invention. That is, the present invention provides an endermic liniment composition, such as various cosmetics, containing 3-methyl-3-methoxybutanol and 1,2-pentanediol or 2-phenoxy ethanol, which has superior antiseptic properties as well as superior usability and safety.

### DETAILED DESCRIPTION OF THE INVENTION

In this invention, “an endermic liniment composition” includes all compositions used for endermic use; for example it includes compositions which can be used widely in cosmetics such as foundation cosmetics, makeup cosmetics, hair cosmetics, etc. as well as in various drugs and/or quasi drugs such as ointments. The present invention also provides these modes of endermic liniment composition individually.

In the present invention, “antiseptic” means resistance against all contaminating microorganisms such as bacteria, fungi, yeast, etc., and “antiseptic effectiveness” means protection against all these contaminating microorganisms. Therefore, even when only the word “antiseptic” is used in the present specifications, the concept of “antimildew” is not excluded.

Embodiments of the present invention are described below.

A. The effective component of the antiseptic agent of the present invention and specific embodiments:

2,2-dialkyl-1,3-propanediol which is used as an effective component of the antiseptic agent of the present invention is a neopentyl-type 1,3-propanediol represented by the above formula (I) [hereafter this 2,2-dialkyl-1,3 -propanediol (I) is also referred to as “compound (I)”].

Different or identical alkyl groups with a carbon number of 1-4 which can be used for R¹ and R² include a methyl group, ethyl group, n-propyl group, isopropyl group, n-butyl group, isobutyl group, secondary butyl group, and tertiary butyl group.

Compound (I) is generally known as an prior art and has been disclosed in general engineering literature and/or patents in many forms. However, usually this is widely used as the raw material of urethane products and as a component of an insect repellent, and there has been no case where this was used as an effective component of an antiseptic agent.

Examples of compound (I) which are particularly superior in their antiseptic effect, less irritating, and superior in terms of usability of endermic liniments containing them include those whose R¹ and R² are a ethyl group, n-propyl group, isopropyl group, or n-butyl group (R¹ and R² can be either identical or different from each other). In particular, 2-n-butyl-2-ethyl-1,3-propanediol is easy to obtain and synthesize at the time of filing this application. Therefore, considering both the practical and economical aspects of implementation of the present invention, 2-n-butyl-2-ethyl-1,3-propanediol is superior as an effective component of the antiseptic agent of the present invention.

Compound (I) other than 2-n-butyl-2-ethyl-1,3-propanediol can also be prepared according to a prior art method, and such a product can be used for an effective component of the antiseptic agent of the present invention. Some examples of compound (1), including 2-n-butyl-2-ethyl-1,3-propanediol, are commercially available (products from Kyowa Hakko K.K., for example), and these commercial products can also be used as an effective component of the antiseptic agent of the present invention.

Compound (I) can either be used as it is for the antiseptic agent of the present invention or diluted/extended by using a diluent, filler or such.

As discussed later, compound (I) is preferably used in combination with propylene glycol and/or diols in an endermic liniment composition, and therefore both compound (I) and propylene glycol and/or diols (specific details will be given below) can be added to the antiseptic agent of the present invention. The form of the antiseptic agent of the present invention can be chosen as appropriate, as long as the effect of compound (I) in the original antiseptic agent of the present invention is not affected.

As described above, the antiseptic agent of the present invention with superior antiseptic effectiveness and superior safety is thus provided.

B. Embodiments of the endermic liniment composition of the present invention:

The endermic liniment composition of the present invention is an endermic liniment composition which contains the antiseptic agent of the present invention as described above.

The blend ratio of the antiseptic agent of the present invention in the endermic liniment composition of the present invention is, for full manifestation of the desired antiseptic effect in the endermic liniment composition, preferably 0.05 wt % or more, more preferably 0.5 wt % or more, in compound (I) equivalent, of the total amount of the composition (hereafter, the blend ratio of the antiseptic agent of the present invention will be expressed in compound (I) equivalent, unless specified otherwise). The antiseptic effect can be significantly increased by blending in 1.0 wt % or in ore of the total amount of the composition.

When the blend ratio of the antiseptic agent of the present invention is 1.5 wt % or more, a superior antiseptic effect can be achieved essentially without using antiseptic components other than compound (I) which is an effective component of the antiseptic agent of the present invention such as paraoxy benzoate (commonly called parabens), salcylic acid and sorbic acid or antiseptic assistants such as phenoxy ethanol (“essentially without using other antiseptic components” means either antiseptic components other than compound (I) are not used at all, or the antiseptic effect of the other antiseptic component is latent in the endermic liniment composition (an example is a case when a compound which can be used for the other antiseptic component is used for a purpose unrelated to its antiseptic effect)).

The upper limit of the blend ratio of the antiseptic agent of the present invention in the endermic liniment composition of the present invention should not be limited in particular. However, usually if the blend ratio is more than 3.0 wt % of the total amount of the composition then the skin sensation at the time of use tends to become heavy; and, if it is more than 10.0 wt %, then compound (I)'s characteristic odor becomes conspicuous and the quality of the endermic liniment composition tends to be degraded.

Also, diols such as propylene glycol, 1,3-butylene glycol, 1,2-pentanediol, dipropylene glycol, 1,2-butylene glycol, 2,5-hexane diol, 2,4-pentanediol, 2-methyl-2,4-pentanediol, 1,2-hexylene glycol, 1,6-hexylene glycol, and 1,5-pentanediol, of which the most preferable is 1,3-butylene glycol, can be used in combination with compound (I) in the endermic liniment composition of the present invention. In this case, even when the blend ratio of compound (I) in the endermic liniment composition is relatively low, adequate antiseptic properties are surprisingly ensured without adding antiseptic agents such as paraben or antiseptic assistants such as phenoxy ethanol, and an endermic liniment composition superior in both usability and safety is provided.

Particularly, the endermic liniment composition of the present invention with this combination of components tends to be superior in usability and thus preferable. When this combination of components is used, the blend ratio of the aforementioned diol in the endermic liniment composition of the present invention is preferably 0.1 -15 wt % of the total amount of the composition and 0.1 -20 times (weight ratio) the amount of compound (I).

The present invention does not exclude the addition of other antiseptic components and/or antiseptic assistant components to the endermic liniment composition of the present invention as desired, even if compound (I) can provide adequate antiseptic effectiveness to the endermic liniment composition of the present invention and there is no need to add other antiseptic components and/or antiseptic assistant component.

(1) 3-methyl-3-methoxybutanol, which is one of the two essential components blended in the endermic liniment composition (hereafter referred to as “the present invention's endermic liniment composition”), has a structure represented by the following formula.

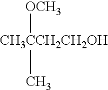

This compound is a component normally blended in an endermic liniment composition as one of the solvents of perfumes. It can be prepared with a common prior art method and blended in the present invention's endermic liniment composition. Commercially available products (such as those from Kuraray Co., Ltd.) can also be blended in the present invention's endermic liniment composition.

The blend ratio of 3-methyl-3-methoxybutanol in the present invention's endermic liniment composition is not limited in particular, and can be determined as appropriate depending on the required degree of antiseptic effectiveness and the blend ratio of 1,2-pentanediol which is used in combination with it. In order to effectively in manifest the desired antiseptic effect in the endermic liniment composition, 0.1 wt % or more of the total composition is a preferable blend ratio. The upper limit of the blend ratio is not limited in particular; 10.0 wt % of the total amount of the composition is sufficient and no improvement in the effect can be expected by increasing it further.

(2) 1,2-pentanediol, which is another essential component to be blended in the present invention's endermic liniment composition along with the aforementioned 3-methyl-3-methoxybutanol, is a compound with a structure represented by the following formula.
CH₃(CH₂ )₂CH(OH)CH₂OH

This 1,2-pentanediol is a component normally blended in an endermic liniment composition as one of the humectants. For the 1,2-pentanediol to be blended in the present invention's endermic liniment composition, those prepared with a normal prior method can be used. Commercially available products (such as those from DRAGOCO) can also be used.

The blend ratio of 1,2-pentanediol in the present invention's endermic liniment composition is not limited in particular, and can be determined as appropriate depending on the required degree of antiseptic effectiveness and the blend ratio of 3-methyl-3-methoxybutanol which is used in combination with it. In order to effectively manifest the desired antiseptic effect in the endermic liniment composition, 0.1 wt % or in more of the total composition is a preferable blend ratio.

The upper limit of the blend ratio of this 1,2-pentanediol should be decided as appropriate depending on the nature of the endermic liniment composition and should not limited in particular; 10.0 wt % of the total amount of the composition is sufficient and no improvement in the effect can be expected by increasing it further. Blending 20.0 wt % or more of the total amount of the endermic liniment composition is not preferable because then the usability of the endermic liniment composition is affected due to stickiness and such.

For the relative blend ratio between the aforementioned 3-methyl-3-methoxybutanol and 1,2-pentanediol which are used in the present invention's endermic liniment composition as an antiseptic means, it is preferable if the blend ratio of one is smaller when the blend ratio of the other is larger, or vice versa, for the purpose of effectively manifesting the intended effect of the present invention.

For example, as shown in Examples later, when the blend ratio of 3-methyl-3-methoxybutanol is 0.1 wt % or less (excluding 0 wt %) of the total amount of the composition, the result is relatively good if 7.0 wt % or more of 1,2-pentanediol is blended in. When the blend ratio of 3-methyl-3-methoxybutanol is more than 0.1 wt % and 2.0 wt % or less of the total amount of the composition, the result is relatively good if 3.0 wt % or more of 1,2-pentanediol is blended in. When the blend ratio of 3-methyl-3-methoxybutanol is more than 2.0 wt % and 4.0 wt % or less of the total amount of the composition, the result is relatively good if 2.0 wt % or more of 1,2-pentanediol is blended in.

When the blend ratio of 3-methyl-3-methoxybutanol is more than 4.0 wt % and 7.0 wt % or less of the total amount of the composition, the result is relatively good if 1.0 wt % or more of 1,2-pentanediol is blended in. When the blend ratio of 3-methyl-3-methoxybutanol is more than 7.0 wt % and 10.0 wt % or less of the total amount of the composition, the result is relatively good if 0.1 wt % or more of 1,2-pentanediol is blended in.

(3) 2-phenoxy ethanol, which is another essential component to be blended in the present invention's endermic liniment composition along with the aforementioned 3-methyl-3-methoxybutanol, is a compound with a structure represented by the following formula. This 2-phenoxy ethanol is a component normally blended in an endermic liniment composition as an antiseptic assistant.

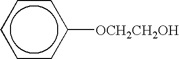

For the 2-phenoxy ethanol to be blended in the present invention's endermic liniment composition, those prepared with a normal prior method, such as a method in which phenol is made to react with ethylene oxide or a method which uses a reaction between sodium phenoxide and ethylene chlorohydrine, can be used. Commercially available products can also be used.

The blend ratio of 2-phenoxy ethanol in the present invention's endermic liniment composition is not limited in particular, and can be determined as appropriate depending on the required degree of antiseptic effectiveness and the blend ratio of 3-methyl-3-methoxybutanol which is used in combination with it. In order to effectively manifest the desired antiseptic effect in the endermic liniment composition, 0.01 wt % or more of the total composition is a preferable blend ratio.

The upper limit of the blend ratio of this 2-phenoxy ethanol should be decided as appropriate depending on the nature of the endermic liniment composition and should not limited in particular. One of the features of the present invention is that the blend ratio of 2-phenoxy ethanol can be reduced. Therefore, the blend ratio of 2-phenoxy ethanol is preferably small in consideration for some users who are sensitive to phenoxy ethanol, as long as the intended effect of the present invention is not affected.

For the relative blend ratio between the aforementioned 3-methyl-3-methoxybutanol and 2-phenoxy ethanol which are used in the present invention's endermic liniment composition as an antiseptic means, it is preferable if the blend ratio of one is smaller when the blend ratio of the other is larger, or vice versa, for the purpose of effectively manifesting the intended effect of the present invention.

For example, as shown in Examples later, when the blend ratio of 3-methyl-3-methoxybutanol is 0.1 wt % or less (excluding 0 wt %) of the total amount of the composition, the result is relatively good if 0.5 wt % or more of 2-phenoxy ethanol is blended in. When the blend ratio of 3-methyl-3-methoxybutanol is more than 0.1 wt % and 1.0 wt % or less of the total amount of the composition, the result is relatively good if 0.3 wt % or more of 2-phenoxy ethanol is blended in. When the (blend ratio of 3-methyl-3-methoxybutanol is more than 1.0 wt % and 3.0 wt % or less, the result is relatively good if 0.1 wt % or more of 2-phenoxy ethanol is blended in.

When the blend ratio of 3-methyl-3-methoxybutanol is more than 3.0 wt % and 5.0 wt % or less of the total amount of the composition, the result is relatively good if 0.05 wt % or more of 2-phenoxy ethanol is blended in. When the blend ratio of 3-methyl-3-methoxybutanol is more than 5.0 wt % and 10.0 wt % or less of the total amount of the composition, the result is relatively good if 0.01 wt % or more of 2-phenoxy ethanol is blended in.

(4) As described above, according to the present invention, by using a new combination of 3-methyl-3 -methoxybutanol and 1,2-pentanediol or 2-phenoxy ethanol, an endermic liniment composition can be provided which ensures adequate antiseptic properties and has both superior usability and safety, to our surprise, by using a small amount of 2-phenoxy ethanol without additionally blending in antiseptic agents such as parabens.

In a preferred embodiment a method is provided for preserving a cosmetic which comprises mixing therein a substantially paraben-free endermic liniment composition comprising an antiseptic/antifungal agent for inhibition of microbial growth in cosmetics. The preferred agent comprises

0.1-3 wt % of 2-n-butyl-2-ethyl-1,3-propanediol,

2.0-5.0 wt % of 1,3-butylene glycol, and

water.

This method is preferred for preserving moisturizing creams comprising 40-60 wt % of water.

In another preferred embodiment a method is provided for preserving a cosmetic which comprises mixing there in an antiseptic/antifungal agent. A preferred agent comprises

0.1-3.0 wt % of 2,2-dialkyl-1,3-propanediol; and

0.1-15.0 wt % of a diol selected from the group consisting of propylene glycol, 1,3-butylene glycol, 1,2-pentanediol, dipropylene glycol, 1,2-butylene glycol, 2,5-pentanediol,2,4-pentanediol,2-methyl-2,5-pentanediol, 1,2-hexylene glycol, and 1,6-hexylene glycol.

3: Common Description:

Depending on the specific embodiment of the endermic liniment composition, components normally blended in an endermic liniment composition can be blended into the endermic liniment composition of the present invention within the range which does not affect the expected effect of the present invention; such components include humectants, ultraviolet light absorbents, vitamins, animal/plant extracts, anti-inflammatories, whiteners, vasodilators, astringents, refreshers, and hormones.

As described above, the endermic liniment composition of the present invention can be used widely in product forms for application on skin such as cosmetics, drugs, and quasi drugs, and also a wide variety of formulations are possible, such as the aqueous solution system, solubilized system, emulsion system, oil-liquid system, gel system, paste system, ointment system, □erosol system, water-oil two layer system, water-oil-powder three layer system. That is, in terms of basic cosmetics, it can be used widely in the various formulations as described above and in forms such as a cleansing cosmetic, lotion, emulsion, cream, gel, essence, and pack/mask. In terms of hair cosmetics, it can be used widely in the various formulations as described above and in formins such as a shampoo, rinse, hair dressing, and hair restoration cosmetic.

In terms of drugs and quasi drugs, it can be widely used in the forms of various types of ointment, for example. Potential formulations and forms of the endermic liniment composition of the present invention are not limited to these formulations and formins.

Depending on the aforementioned desired formulations and formins, usual prior art base components can be widely blended in the endermic liniment composition of the present invention, as long as the expected effect of the present invention is not affected by this blending. That is, appropriate amounts of liquid fats/oils, solid fats/oils, waxes, hydrocarbon oils, higher fatty acids, higher alcohols, synthetic ester oils, silicones, various surfactants, sequestering agents, water soluble polymers, thickeners, various powder components, colorings, perfumes, and water can be blended as required into the endermic liniment composition of the present invention.

Specific recipes of the endermic liniment composition of the present invention are described below in the Examples section.

### EXAMPLES

The present invention is described in detail by referring to the examples below. The technical scope of the present invention is not limited to these examples.

The blend ratios indicated by “wt %” or “%” in the examples are in weight percent units of the total amount of that into which the components were blended, unless specified otherwise.

Before disclosing the examples, the actual usage test and the antiseptic effectiveness evaluation test are described.

The Actual Usage Test:

A panel of 30 people who had complained about skin irritation when using endermic liniment compositions containing paraben, used the endermic liniment compositions of the present invention and others twice a day, morning and evening, for one week, and reported the degree of satisfaction in terms of usability and presence/absence of skin irritation.

The Antiseptic Effectiveness Evaluation Test:

(Preservative Effect)

30 ml of the sample was inoculated with microbe-containing fluid, and the change in the number of microbes was checked with the smearing method.

Mold, yeast, and bacteria were used as the inoculation microbes. The antiseptic effectiveness was evaluated based on the changes in the number of the microbes in two to four weeks, and the obtained results were classified by using the following four step criterion. Of the following classes, □ and ∘ were defined as acceptable.

□: A 99.9% or more decrease in microbial concentration was observed within 2 weeks; for yeast, a 99.9% or more decrease was observed within 2 weeks; and for mold, a 90% or more decrease was observed within 2 weeks.

∘: A 99.9% or more decrease in microbial concentration was observed within 3 weeks; for yeast, a 99.9% or more decrease was observed within 3 weeks; and for mold, a 90% or more decrease was observed within 3 weeks.

□: A 99.9% or more decrease in microbial concentration was observed within 4 weeks; for yeast, a 99.9% or more decrease was observed within 4 weeks; and for mold, a 90% or more decrease was observed within 4 weeks.

×: For bacteria, concentration decreased less than 99.9 % within 4 weeks; of yeast, concentration decreased less than 99% within 4 weeks; and for mold, concentration decreased less than 90% within 4 weeks.

The detailed steps are as follows:

### Examples 1-1-1-4 and Comparative Example 1-1

Using the recipes shown in the following Table 1-1, lotions which were embodiments of the endermic liniment composition of the present invention as well as comparative examples were subjected to the aforementioned ed actual usage test and antiseptic effectiveness evaluation test, and the results were recorded. For the preparation method of these lotions, a method commonly used for preparing lotions was used.

TABLE 1-1
(Blend ratio: wt %)

| | | | | | Comparative |
|---|---|---|---|---|---|
| | Example | | | | example |
| | 1-1 | 1-2 | 1-3 | 1-4 | 1-1 |
| 2-n-butyl-2-ethyl-1,3- | 0.5 | 1.0 | 1.0 | 0.5 | — |
| propanediol | | | | | |
| 1,3-butylene glycol | 2.0 | 2.0 | — | — | 2.0 |
| Ethyl alcohol | 3.0 | 3.0 | 3.0 | 3.0 | 3.0 |
| Glycerine | 1.0 | 1.0 | 1.0 | 1.0 | 1.0 |
| Polyoxyethylene(POE = 60) | 0.4 | 0.4 | 0.4 | 0.4 | 0.4 |
| hydrogenated castor | | | | | |
| oil | | | | | |
| Citric acid | 0.03 | 0.03 | 0.03 | 0.03 | 0.03 |
| Trisodium citrate | 0.07 | 0.07 | 0.07 | 0.07 | 0.07 |
| Trisodium edentate | 0.02 | 0.02 | 0.02 | 0.02 | 0.02 |
| Methyl paraben | — | — | — | 0.2 | — |
| Purified water | Balance to make the total 100 | | | | |
| <Results of the actual | | | | | |
| usage test> | | | | | |
| Those who complained | 0/30 | 0/30 | 0/30 | 14/30 | 0/30 |
| about skin irritation | | | | | |
| Those who were | 29/30 | 29/30 | 28/30 | 26/30 | 28/30 |
| satisfied with the | | | | | |
| usability | | | | | |
| Results of the antiseptic | ◯ | ⊚ | ⊚ | ⊚ | X |
| effectiveness test | | | | | |

With Examples 1-1 and 1-2. the majority of people in the panel reported that skin irritation was minor and usability was satisfactory, and these Examples also showed superior antiseptic effectiveness which is a preservative effect in cosmetics.

Even with Example 1-3, which didn't contain 1,3-butylene glycol, the majority of people in the panel reported that skin irritation was minor, usability was satisfactory, and antiseptic effectiveness was satisfactory. However, with Comparative Example 1-1, which did not contain 2-n-butyl-2-ethyl-1,3-propanediol, although the majority of people in the panel reported that skin irritation was minor and usability was satisfactory, the antiseptic effectiveness thereof was inferior.

With Example 1-4, which contained methyl paraben, although the antiseptic effectiveness was superior, many panelists reported skin irritation such as itching and tingling.

These results clearly indicate that an endermic liniment composition which maintains the antiseptic effectiveness, causes less skin irritation, and has good usability is provided by blending the antiseptic agent of the present invention into the endermic liniment composition such that the blend ratio of 2,2-dialkyl-1,3-propanediol is approximately 0.5 -1%, even when paraben is not blended in.

### Examples 1-5 -1-15

Using lotions with various blend ratios of 2-n-butyl-2-ethyl-1,3-propanediol the aforementioned antiseptic effectiveness test and the actual usage test (only usability) were conducted.

The blend ratio of 2-n-butyl-2-ethyl-1,3-propanediol in each lotion and the test results are shown in Table 1 -2.

TABLE 1-2
(Blend ratio: wt %)

| | Example | | | | | | | | | | |
|---|---|---|---|---|---|---|---|---|---|---|---|
| | | | | | | 1- | 1- | 1- | 1- | 1- | 1- |
| | 1-5 | 1-6 | 1-7 | 1-8 | 1-9 | 10 | 11 | 12 | 13 | 14 | 15 |
| 2-n-butyl-2-ethyl- | 0.05 | 0.1 | 0.5 | 1.0 | 2.0 | 3.0 | 5.0 | 10.0 | 0.5 | 2.0 | 10.0 |
| 1,3-propanediol | | | | | | | | | | | |
| 1,3-butylene | 5.0 | 5.0 | 5.0 | 4.0 | 4.0 | 4.0 | 4.0 | ‘2.0 | — | — | — |
| glycol | | | | | | | | | | | |
| <Results of the | | | | | | | | | | | |
| actual usage test> | | | | | | | | | | | |
| Those who were | 29/30 | 28/30 | 28/30 | 29/30 | 23/30 | 16/30 | 10/30 | 2/30 | 27/30 | 15/30 | 0/30 |
| satisfied with the | | | | | | | | | | | |
| usability | | | | | | | | | | | |
| Results of the | Δ | ◯ | ◯ | ⊚ | ⊚ | ⊚ | ⊚ | ⊚ | Δ | ⊚ | ⊚ |
| antiseptic | | | | | | | | | | | |
| effectiveness | | | | | | | | | | | |
| test | | | | | | | | | | | |

compositions of the present invention exhibited adequate antiseptic effectiveness when they contained the antiseptic agent of the present invention such that the concentration of 2-n-butyl-2-ethyl-1,3-propanediol was 0.5% or more. Even at 0.1%, however, an adequate antiseptic effect on bacteria was confirmed, if 1,3-butylene glycol was additionally blended into the endermic liniment composition. The comprehensive evaluation confirmed that 0.5% or more was desirable, in order to have an effect on all of the mold, yeast, and bacteria.

However, for products with a lower water content of 40 -60%, such as nourishing cream, even a blend ratio of 0.3% is expected to have an adequate effect, because the concentration of 2-n-butyl-2-ethyl-1,3-propanediol in the water phase increases. On the other hand, when the blend ratio of the antiseptic agent of the present invention in the endermic liniment composition was 3 wt % or more in 2-n-butyl-2-ethyl-1,3-propanediol equivalent, although there was no problem in terms of the antiseptic effect, the usability of the composition was degraded, as the liniment became heavy, and the panel's response became negative.

These results confirm that by blending in a small amount, in 2-n-butyl-2-ethyl-1,3-propanediol equivalent, of the antiseptic a gent of the present invention, an endermic liniment composition with superior antiseptic effectiveness and good skin sensation during use can be provided without blending in antiseptic agents such as paraben or by reducing the amount of antiseptic agents.

Those which did not contain 1,3-butylene glycol (Examples 1-13-1-15) showed superior antiseptic effectiveness by blending in the antiseptic agent of the present invention, but their usability clearly decreased when the amount of the antiseptic agent of the present invention was increased to further improve the antiseptic effectiveness. On the other hand, when 1,3-butylene glycol was added in combination, not only did this addition contribute to the antiseptic effectiveness but the usability was also improved.

Examples of endermic liniment compositions of the present invention with various recipes are shown below. All Examples had less skin irritation and good usability while maintaining superior antiseptic effectiveness. For the method of preparing endermic liniment compositions of these Examples, commonly used methods for preparing endermic liniment compositions of each embodiment were followed. The amount of water was adjusted such that the total amount would be 100.

### Example 1-16

### Astringent Lotion

| Component in the composition | Blend ratio (wt %) |
|---|---|
| 2-n-butyl-2-ethyl-1,3-propanediol | 1.0 |
| Polyoxyethylene (POE = 50) oleyl ether | 0.5 |
| Polyethylene glycol 300 | 1.0 |
| Ethyl alcohol | 8.0 |
| Glycerine | 3.0 |
| Lactic acid | 0.02 |
| 50% aqueous solution of sodium lactate | 0.25 |
| Trisodium edetate | 0.1 |
| Purified water | Balance |

### Example 1 -17

### Astringent Lotion

| Component in the composition | Blend ratio (wt %) |
|---|---|
| 2,2-diethyl-1,3-propanediol | 1.0 |
| Polyoxyethylene (POE = 50) oleyl ether | 0.5 |
| Polyethylene glycol 300 | 1.0 |
| Ethyl alcohol | 8.0 |
| Glycerine | 3.0 |
| Lactic acid | 0.02 |
| 50% aqueous solution of sodium lactate | 0.25 |
| Trisodium edetate | 0.1 |
| Purified water | Balance |

### Example 1-18

### Cleansing Foam

| Component in the composition | Blend ratio (wt %) |
|---|---|
| Stearic acid | 8.0 |
| Palmitic acid | 6.0 |
| Myristic acid | 6.0 |
| Lauric acid | 4.0 |
| Potassium hydroxide | 5.2 |
| Glyceryl monostearate | 2.0 |
| Beeswax | 1.5 |
| 2,2-di-n-butyl-1,3-propanediol | 0.6 |
| 1,2-pentanediol | 1.0 |
| Polyethylene glycol 1500 | 5.0 |
| Glycerine | 10.0 |
| Purified water | Balance |

### Example 1-19

### Emollient Emulsion

| Component in the composition | Blend ratio (wt %) |
|---|---|
| 2-n-propyl-2-n-butyl-1,3-propanediol | 0.1 |
| Glycerine | 5.0 |
| Cetanol | 1.5 |
| Stearyl alcohol | 1.8 |
| Petrolatum | 2.0 |
| Dimethylpolysiloxane (20 cs) | 1.5 |
| Squalane | 2.5 |
| Isopropyl myristate | 2.5 |
| Glyceryl monostearate | 1.8 |
| Polyoxyethylene (POE = 5) glyceryl monostearate | 1.8 |
| Polyoxyethylene (POE = 20) cetyl ether | 1.5 |
| Carboxyvinyl polymer | 0.25 |
| Potassium hydroxide | 0.05 |
| L-arginine | 0.2 |
| Xylitol | 2.0 |
| Dipropylene glycol | 2.0 |
| 1,3-butylene glycol | 3.0 |
| Trisodium edetate | 0.02 |
| Purified water | Balance |

### Example 1-20

### Skin Treatment Gel

| Component in the composition | Blend ratio (wt %) |
|---|---|
| 2,2-di-n-propyl-1,3-propanediol | 1.0 |
| Dimethylpolysiloxane | 0.5 |
| Isopropyl myristate | 1.5 |
| Polyoxyethylene (POE = 60) | 0.5 |
| hydrogenated castor oil | |
| Tocopherol acetate | 0.2 |
| Monoammonium glycyrrhizate | 0.05 |
| Carboxyvinyl polymer | 0.45 |
| Potassium hydroxide | 0.15 |
| Glycerine | 12.0 |
| Dipropylene glycol | 2.0 |
| Trisodium edetate | 0.01 |
| Purified water | Balance |

### Example 1-21

### Moisture Cream

| Component in the composition | Blend ratio (wt %) |
|---|---|
| 2-n-propyl-2-isopropyl-1,3-propanediol | 1.0 |
| Stearyl alcohol | 5.5 |
| Stearic acid | 2.0 |
| Squalane | 12.5 |
| Isopropyl myristate | 7.5 |
| Polyoxyethylene (POE = 25) cetyl alcohol ether | 3.0 |
| Glyceryl monostearate | 2.0 |
| Tocopherol acetate | 0.2 |
| Monoammonium glycyrrhizate | 0.05 |
| Glycerine | 5.0 |
| Dipropylene glycol | 2.0 |
| Trisodium edetate | 0.01 |
| Purified water | Balance |

### Example 1-22

### Essence

| Component in the composition | Blend ratio (wt %) |
|---|---|
| 2-isopropyl-2-ethyl-1,3-propanediol | 1.0 |
| Dimethylpolysiloxane | 0.1 |
| Olive oil | 0.2 |
| Polyoxyethyleneoleyl alcohol ether | 1.0 |
| Tocopherol acetate | 0.1 |
| Ethanol | 6.5 |
| Hyaluronic acid | 0.1 |
| Sorbitol | 8.0 |
| Dipropylene glycol | 2.0 |
| Trisodium edetate | 0.01 |
| Purified water | Balance |

### Example 1-23

### Essence

| Component in the composition | Blend ratio (wt %) |
|---|---|
| 2-n-propyl-2-ethyl-1,3-propanediol | 1.0 |
| Dimethylpolysiloxane | 0.1 |
| Olive oil | 0.2 |
| Polyoxyethyleneoleyl alcohol ether | 1.0 |
| Tocopherol acetate | 0.1 |
| Ethanol | 6.5 |
| Hyaluronic acid | 0.1 |
| Sorbitol | 8.0 |
| Dipropylene glycol | 2.0 |
| Trisodium edetate | 0.01 |
| Purified water | Balance |

### Example 1-24

### Jelly Pack

| Component in the composition | Blend ratio (wt %) |
|---|---|
| 2,2-diisopropyl-1,3-propanediol | 1.0 |
| Polyoxyethyleneoleyl alcohol ether | 0.5 |
| Monoammonium glycyrrhizate | 0.05 |
| Carboxymethyl cellulose | 5.0 |
| Ethanol | 12.0 |
| Polyvinyl alcohol | 12.0 |
| 1,3-butylene glycol | 5.0 |
| Trisodium edetate | 0.01 |
| Purified water | Balance |

### Example 1-25

### Eye Liner

| Component in the composition | Blend ratio (wt %) |
|---|---|
| 2-isopropyl-2-n-butyl-1,3-propanediol | 0.6 |
| Iron oxide (black) | 14.0 |
| Isopropyl myristate | 1.5 |
| Polyoxyethylenesorbitan monooleate | 1.0 |
| Vinyl acetate resin emulsion | 45.0 |
| Monoammonium glycyrrhizate | 0.05 |
| Carboxyvinyl polymer | 1.5 |
| Acetyltributyl citrate | 1.0 |
| Glycerine | 5.0 |
| Dipropylene glycol | 2.0 |
| Trisodium edetate | 0.01 |
| Purified water | Balance |

### Examples 2-1, 2-2 and

### Comparative examples 2-1 -2-3

Using the recipes shown in the following Table 2-1, lotions of Examples 2-1 and 2-2, as an embodiment of the present invention's endermic liniment composition, and lotions of Comparative examples 2-1, 2-2, and 2-3 were subjected to the aforementioned test of usability and such and antiseptic effectiveness evaluation test, and the results are listed in Table 2-1 as well. For the preparation method of these lotions, a method commonly used for preparing lotions was used.

TABLE 2-1
(Blend ratio: wt %)

| Example | | Comparative example | | |
|---|---|---|---|---|
| 2-1 | 2-2 | 2-1 | 2-2 | 2-3 |

| 3-methyl-3- | 1.0 | 3.0 | 3.0 | — | 3.0 |
|---|---|---|---|---|---|
| methoxybutanol | | | | | |
| 1,2-pentanediol | 3.0 | 3.0 | — | 3.0 | 3.0 |
| Ethyl alcohol | 2.0 | 2.0 | 2.0 | 2.0 | 2.0 |
| Glycerine | 1.0 | 1.0 | 1.0 | 1.0 | 1.0 |
| Polyoxyethylene (POE = 60) | 0.4 | 0.4 | 0.4 | 0.4 | 0.4 |
| hydrogenated | | | | | |
| castor oil | | | | | |
| Citric acid | 0.03 | 0.03 | 0.03 | 0.03 | 0.03 |
| Trisodium citrate | 0.07 | 0.07 | 0.07 | 0.07 | 0.07 |
| Trisodium edetate | 0.02 | 0.02 | 0.02 | 0.02 | 0.02 |
| Methyl paraben | — | — | — | — | 0.2 |
| Purified water | Balance to make the total 100 | | | | |
| <Results of the actual | | | | | |
| usage test> | | | | | |
| Those who complained | 0/30 | 0/30 | 0/30 | 0/30 | 21/30 |
| about skin irritation | | | | | |
| Those who were | 29/30 | 28/30 | 28/30 | 29/30 | 26/30 |
| satisfied with the | | | | | |
| usability | | | | | |
| Antiseptic | ◯ | ⊚ | X | X | ⊚ |
| effectiveness test | | | | | |

As indicated in the results shown in Table 2-1, with Examples 1 and 2, the majority of people in the panel reported that skin irritation was minor and usability was satisfactory. These Examples further illustrate the superior antiseptic effectiveness of the present invention. On the other hand, with Comparative Example 2-1, which did not contain 1,2-pentanediol, although the majority of people in the panel reported that skin irritation was minor and usability was satisfactory, the antiseptic effectiveness was inferior Further, with Comparative Example 2-2, which did not contain 3-methyl-3-methoxybutanol, although the majority of people in the panel reported that skin irritation was minor and usability was satisfactory, the antiseptic effectiveness was inferior.

With Comparative Example 2-3, which contained methyl paraben, although the antiseptic effectiveness was superior, many panelists reported skin irritation. These results clearly indicate that the combination of 3-methyl-3-methoxybutanol and 1,2-pentanediol can provide an endermic liniment composition which shows less skin irritation and good usability while maintaining the antiseptic effectiveness.

### Examples 2-3 -2-8 and

### Comparative Examples 2-4 and 2-5

Following these Examples and Comparative Examples, the aforementioned antiseptic effectiveness test and the usability test were conducted on lotions with varied blend ratios of 3-methyl-3-methoxybutanol and 1,2-pentanediol (the components and their blend ratios other than 3-methyl-3-methoxybutanol and 1,2-pentanediol are the same as those shown in the aforementioned Table 2-1). The blend ratios of 3-methyl-3-methoxybutanol and 1,2-pentanediol in each lotion and the test results are shown in Table 2-2.

TABLE 2-2
(Blend ratio: wt %)

| | | | | | | | Comparative | |
|---|---|---|---|---|---|---|---|---|
| | Examples | | | | | | examples | |
| | 2-3 | 2-4 | 2-5 | 2-6 | 2-7 | 2-8 | 2-4 | 2-5 |
| 3-methyl-3- | 0.1 | 2.0 | 4.0 | 7.0 | 10.0 | 10.0 | 4.0 | 10.0 |
| methoxy- | | | | | | | | |
| butanol | | | | | | | | |
| 1,2-pentanediol | 7.0 | 3.0 | 2.0 | 1.0 | 0.1 | 0.5 | — | — |
| <Usability test | | | | | | | | |
| results> | | | | | | | | |
| those who are | 28/30 | 29/30 | 30/30 | 28/30 | 30/30 | 29/30 | 29/30 | 29/30 |
| satisfied with | | | | | | | | |
| usability | | | | | | | | |
| Antiseptic | ◯ | ◯ | ◯ | ◯ | ◯ | ⊚ | X | Δ |
| effectiveness | | | | | | | | |

According to the test results shown in Table 2 -2, Example 2 -3, which had 0.1 wt % of 3-methyl-3-methoxybutanol showed satisfactory results with 7.0 wt % of 1,2-pentanediol. Example 2-4, which had 2.0 wt % of 3-methyl-3-methoxybutanol, showed satisfactory results with 3.0 wt % of 1,2-pentanediol. Example 2-5, which had 4.0 wt % of 3-methyl-3-methoxybutanol, showed satisfactory results with 2.0 wt % of 1,2-pentanediol.

Also, Example 2-6, which had 7.0 wt % of 3-methyl-3-methoxybutanol, showed satisfactory results with 1.0 wt % of 1,2-pentanediol. Examples 2-7 and 2-8, which had 10 wt % of 3-methyl-3-methoxybutanol, showed satisfactory results in terms of both usability and antiseptic effectiveness with 0.1 wt % (Example 2-7) and 0.5 wt % (Example 2-8) of 1,2-pentanediol. Example 2-8, which had a higher blend ratio of 1,2-pentanediol, showed particularly superior antiseptic effectiveness.

Comparative examples 2-4 and 2-5, which did not contain any 1,2-pentanediol, both showed inadequate antiseptic effectiveness. These test results also clearly indicated that not only the amount of 3-methyl-3-methoxybutanol and the amount of 1,2-pentanediol blended in the endermic liniment composition of the present invention but also their relative blend ratio influences the intended effect of the present invention.

In terms of the blend ratios of 3-methyl-3-methoxybutanol and 1,2-pentanediol in the present invention's endermic liniment composition, these results clearly indicate that, when the blend ratio of 3-methyl-3-methoxybutanol is 0.1 wt % or less (excluding 0 wt %), the result is relatively good if 7.0 wt % or more of 1,2-pentanediol is blended in. It was also indicated that, when the blend ratio of 3-methyl-3-methoxybutanol is more than 0.1 wt % and 2.0 wt % or less, the result is relatively good if 3.0 wt % or more of 1,2-pentanediol is blended in.

It was also indicated that, when the blend ratio of 3-methyl-3-methoxybutanol is more than 2.0 wt % and 4.0 wt % or less, the result is relatively good if 2 wt % or more of 1,2-pentanediol is blended in. It was also indicated that, when the blend ratio of 3-methyl-3-methoxybutanol is more than 4.0 wt % and 7.0 wt % or less, the result is relatively good if 1.0 wt % or more of 1,2-pentanediol is blended in. Furthermore, it was indicated that, when the blend ratio of 3-methyl-3-methoxybutanol is more than 7.0 wt % and 10.0 wt % or less, the result is relatively good if 0.1 wt % or more of 1,2-pentanediol is blended in.

Examples of endermic liniment compositions of the present invention with various recipes are shown below. All Examples had less skin irritation and good usability while maintaining superior antiseptic effectiveness. For the method of preparing endermic liniment compositions of these Examples, commonly used methods for preparing endermic liniment compositions of each embodiment were followed.

### Example 2-9

### Astringent Lotion

| | wt % |
|---|---|
| 3-methyl-3-methoxybutanol | 1.0 |
| 1,2-pentanediol | 3.0 |
| Polyoxyethylene (POE = 50) oleyl ether | 0.5 |
| Polyethylene glycol 300 | 1.0 |
| Ethyl alcohol | 8.0 |
| Dipropylene glycol | 2.0 |
| Lactic acid | 0.02 |
| 50% aqueous solution of sodium lactate | 0.25 |
| Trisodium edetate | 0.01 |
| Purified water | Balance |

### Example 2-10

### Emollient Emulsion

| | wt % |
|---|---|
| 3-methyl-3-methoxybutanol | 0.1 |
| 1,2-pentanediol | 5.0 |
| Cetanol | 1.5 |
| Stearyl alcohol | 1.8 |
| Petrolatum | 2.0 |
| Dimethylpolysiloxane (20 cs) | 1.5 |
| Squalane | 2.5 |
| Isopropyl myristate | 2.5 |
| Glyceryl monostearate | 1.8 |
| Polyoxyethylene (POE = 5) glyceryl monostearate | 1.8 |
| Polyoxyethylene (POE = 20) cetyl ether | 1.5 |
| Carboxyvinyl polymer | 0.25 |
| Potassium hydroxide | 0.05 |
| L-arginine | 0.2 |
| Glycerine | 4.0 |
| Dipropylene glycol | 1.0 |
| 1,3-butylene glycol | 2.0 |
| Trisodium edetate | 0.02 |
| Purified water | Balance |

### Example 2-11

### Skin Treatment Gel

| | wt % |
|---|---|
| 3-methyl-3-methoxybutanol | 1.0 |
| 1,2-pentanediol | 3.0 |
| Dimethylpolysiloxane | 0.5 |
| Isopropyl myristate | 1.5 |
| Polyoxyethylene (POE = 60) | 0.5 |
| hydrogenated castor oil | |
| Tocopherol acetate | 0.2 |
| Monoammonium glycyrrhizate | 0.05 |
| Carboxyvinyl polymer | 0.45 |
| Potassium hydroxide | 0.15 |
| Glycerine | 16.0 |
| Dipropylene glycol | 2.0 |
| Trisodium edetate | 0.01 |
| Purified water | Balance |

### Examples 3-1,3-2 and

### Comparative Examples 3-1,3-2

Using the recipes shown in the following Table 3-1, lotions of Examples 3-1 and 3-2, as an embodiment of the present invention's endermic liniment composition, and lotions of Comparative examples 3-1 and 3-2 were subjected to the aforementioned test of usability and such and antiseptic effectiveness evaluation test, and the results are listed in Table 3-1 as well. For the preparation method of these lotions, a method commonly used for preparing lotions was used.

TABLE 3-1
(Blend ratio: wt %)

| | | | Comparative | |
|---|---|---|---|---|
| | Example | | example | |
| | 3-1 | 3-2 | 3-1 | 3-2 |
| 3-methyl-3-methoxybutanol | 3.0 | 5.0 | 3.0 | 3.0 |
| 2-phenoxy ethanol | 0.5 | 0.5 | — | — |
| Ethyl alcohol | 2.0 | 2.0 | 2.0 | 2.0 |
| Glycerine | 1.0 | 1.0 | 1.0 | 1.0 |
| Polyoxyethylene (POE = 60) | 0.4 | 0.4 | 0.4 | 0.4 |
| hydrogenated castor oil | | | | |
| Citric acid | 0.03 | 0.03 | 0.03 | 0.03 |
| Trisodium citrate | 0.07 | 0.07 | 0.07 | 0.07 |
| Trisodium edentate | 0.02 | 0.02 | 0.02 | 0.02 |
| Methyl paraben | — | — | — | 0.2 |
| Purified water | Balance to make the total 100 | | | |
| <Results of the usability | | | | |
| test> | | | | |
| Those who complained about | 0/30 | 0/30 | 0/30 | 21/30 |
| skin irritation | | | | |
| Those who were satisfied | 29/30 | 28/30 | 28/30 | 26/30 |
| with the usability | | | | |
| Results of the antiseptic | ◯ | ⊚ | X | ⊚ |
| effectiveness test | | | | |

As indicated by the results shown in Table 3-1, with Examples 3-1 and 3-2, the majority of people in the panel reported that skin irritation was minor and usability was satisfactory, and these Examples also showed superior antiseptic effectiveness. On the other hand, with Comparative example 3-1 which did not contain 2-phenoxy ethanol, although the majority of people in the panel reported that skin irritation was minor and usability was satisfactory, the antiseptic effectiveness was inferior when the blend ratio of 3-methyl-3-methoxybutanol was low.

With Comparative example 3-2 which contained methyl paraben but did not contain 2-phenoxy ethanol just as Comparative example 3-1 did not, although the antiseptic effectiveness was superior, many panelists reported skin irritation. These results clearly indicate that the combination of 3-methyl-3-methoxybutanol and 2-phenoxy ethanol can provide an endermic liniment composition which shows less skin irritation and good usability while maintaining the antiseptic effectiveness.

### Examples 3-3 -3-7 and

### Comparative Examples 3-3 and 3-4

Next, the aforementioned antiseptic effectiveness test and the usability test were conducted on lotions with varied blend ratios of 3-methyl-3-methoxybutanol and 2-phenoxy ethanol (the components and their blend ratios other than 3-methyl-3-methoxybutanol and 2-phenoxy ethanol are the same as those shown in the aforementioned Table 3 -1) The blend ratios of 3-methyl-3-methoxybutanol and 2-phenoxy ethanol in each lotion and the test results are shown in Table 3-2.

TABLE 3-2
(Blend ratio: wt %)

| | | | | | | Comparative | |
|---|---|---|---|---|---|---|---|
| | Examples | | | | | examples | |
| | 3-3 | 3-4 | 3-5 | 3-6 | 3-7 | 3-4 | 3-5 |
| 3-methyl-3- | 0.1 | 1.0 | 3.0 | 5.0 | 10.0 | 3.0 | 10.0 |
| methoxybutanol | | | | | | | |
| 2-phenoxy ethanol | 0.5 | 0.3 | 0.1 | 0.05 | 0.01 | — | — |
| <Results of the | | | | | | | |
| usability test> | | | | | | | |
| Those who were | 29/30 | 28/30 | 29/30 | 28/30 | 27/30 | 27/30 | 26/30 |
| satisfied with the | | | | | | | |
| usability | | | | | | | |
| Results of the | ◯ | ◯ | ◯ | ◯ | ◯ | X | X |
| antiseptic | | | | | | | |
| effectiveness test | | | | | | | |

According to the test results shown in Table 3-2. Example 3-3, which had 0.1 wt % of 3-methyl-3-methoxybutanol, showed satisfactory results with 0.5 wt % of 2-phenoxy ethanol. Example 3-4, which had 1.0 wt % of 3-methyl-3-methoxybutanol, showed satisfactory results with 0.3 wt % of 2-phenoxy ethanol. Example 3-5, which had 3.0 wt % of 3-methyl-3-methoxybutanol, showed satisfactory results with 0.1 wt % of 2-phenoxy ethanol.

Also, Example 3-6, which had 5.0 wt % of 3-methyl-3-methoxybutanol, showed satisfactory results with 0.05 wt % of 2-phenoxy ethanol. Examples 3-7, which had 10 wt % of 3-methyl-3-methoxybutanol, showed satisfactory results in terms of both usability and antiseptic effectiveness with 0.01 wt % of 2-phenoxy ethanol. Comparative examples 3-4 and 3-5, which did not contain any 2-phenoxy ethanol, both showed inadequate antiseptic effectiveness.

In terms of the blend ratios of 3-methyl-3-methoxybutanol and 2-phenoxy ethanol in the present invention's endermic liniment composition, these results clearly indicate that, when the blend ratio of 3-methyl-3-methoxybutanol is 0.1 wt % or less (excluding 0 wt %), the result is relatively good if 0.5 wt % or more of 2-phenoxy ethanol is blended in. It was also indicated that, when the blend ratio of 3-methyl-3-methoxybutanol is more than 0.1 wt % and 1.0 wt % or less, the result is relatively good if 0.3 wt % or more of 2-phenoxy ethanol is blended in.

It was also indicated that, when the blend ratio of 3-methyl-3-methoxybutanol is more than 1.0 wt % and 3.0 wt % or less, the result is relatively good if 0.1 wt % or more of 2-phenoxy ethanol is blended in. It was also indicated that, when the blend ratio of 3-methyl-3-methoxybutanol is more than 3.0 wt % and 5.0 wt % or less, the result is relatively good if 0.05 wt % or in more of 2-phenoxy ethanol is blended in. Furthermore, it was indicated that, when the blend ratio of 3-methyl-3-methoxybutanol is more than 5.0 wt % and 10.0 wt % or less, the result is relatively good if 0.01 wt % or more of 2-phenoxy ethanol is blended in.

Examples of endermic liniment compositions of the present invention with various recipes are shown below. All Examples had less skin irritation and good usability while maintaining superior antiseptic effectiveness. For the method of preparing endermic liniment compositions of these Examples, commonly used methods for preparing endermic liniment compositions of each embodiment were followed.

### Example 3-8

### Astringent Lotion

| | wt % |
|---|---|
| 3-methyl-3-methoxybutanol | 1.0 |
| 2-phenoxy ethanol | 0.3 |
| Polyoxyethylene (POE = 50) oleyl ether | 0.5 |
| Polyethylene glycol 300 | 1.0 |
| Ethyl alcohol | 18.0 |
| Dipropylene glycol | 2.0 |
| Lactic acid | 0.02 |
| 50% aqueous solution of sodium lactate | 0.25 |
| Trisodium edetate | 0.01 |
| Purified water | Balance |

### Example 3-9

### Emollient Emulsion

| | wt % |
|---|---|
| 3-methyl-3-methoxybutanol | 5.0 |
| 2-phenoxy ethanol | 0.1 |
| Cetanol | 1.5 |
| Stearyl alcohol | 1.8 |
| Petrolatum | 2.0 |
| Dimethylpolysiloxane (20 cs) | 1.5 |
| Squalane | 2.5 |
| Isopropyl myristate | 2.5 |
| Glyceryl monostearate | 1.8 |
| Polyoxyethylene (POE = 5) glyceryl monostearate | 1.8 |
| Polyoxyethylene (POE = 20) cetyl ether | 1.5 |
| Carboxyvinyl polymer | 0.25 |
| Potassium hydroxide | 0.05 |
| L-arginine | 0.2 |
| Glycerine | 4.0 |
| Dipropylene glycol | 2.0 |
| 1,3-butylene glycol | 3.0 |
| Trisodium edetate | 0.02 |
| Purified water | Balance |

### Example 3-10

### Skin Treatment Gel

| | wt % |
|---|---|
| 3-methyl-3-methoxybutanol | 3.0 |
| 2-phenoxy ethanol | 0.3 |
| Dimethylpolysiloxane | 0.5 |
| Isopropyl myristate | 1.5 |
| Polyoxyethylene (POE = 60) | 0.5 |
| hydrogenated castor oil | |
| Tocopherol acetate | 0.2 |
| Monoammonium glycyrrhizate | 0.05 |
| Carboxyvinyl polymer | 0.45 |
| Potassium hydroxide | 0.15 |
| Glycerine | 16.0 |
| Dipropylene glycol | 2.0 |
| Trisodium edetate | 0.01 |
| Purified water | Balance |

### Example 4

Preparation Step

Twelve compositions of substantially paraben-free endermic liniments were prepared as follows:

Twelve 30 ml samples of cosmetic of the present invention were prepared, each having varying concentrations of 1,2-pentanediol and 3-methyl-3-methoxybutanol, as shown in Table 4-1 below. These cosmetic compositions contained the following additional elements in the following concentrations:

| | |
|---|---|
| Glycerin | 1.0 wt % |
| Ethyl Alcohol | 2.0 wt % |
| Polyoxyethylene (POE-60) | 0.4 wt % |
| hydrogenated castor oil | |
| Citric acid | 0.03 wt % |
| Trisodium citrate | 0.07 wt % |
| Trisodium edetate | 0.02 wt % |
| Purified water | Balance |

Each of these samples, labeled compositions 1-12, respectively, were placed into 12 sterilized 50 ml screw tubes.

Then a sample of microbe containing fluid was prepared in a test tube containing yeast and bacteria suspended in a solution of ion-exchanged water, and a sample of mold was prepared in a test tube containing mold suspended in an aqueous solution of 0.006 wt % dioctyl sodium sulfosuccinate. The concentrations of mold, yeast and bacteria were 3×10⁶ cfu/ml, 3×10⁷ cfu/ml and 3×10⁸ cfu/ml, respectively, wherein cfu/ml is the number of colonies of mold, yeast or bacteria per ml of fluid.

100 μl of microbe containing fluid described above was then alternately injected into each of the 12 cosmetic samples 1-12, so as to produce a concentration therein of mold, yeast or bacteria of 10⁴ cfu/ml, 10⁵ cfu/ml and 10⁶ cfu/ml, respectively.

The 12 samples were then placed in an incubator maintained at a constant temperature, the temperature dependent upon the type of microbe injected into the sample. Specifically, the samples containing mold and yeast were kept at 25° C., and the bacteria was kept at 30° C.

Testing Step:

The 12 samples described above, and listed numerically as compositions 1-12, were allowed to sit for 2 weeks in incubators as described above. Then, 1 ml of each of the samples was extracted therefrom in to provide a test sample. Further, for the bacteria sample, 4 additional diluted samples of each of the 12 original samples were prepared, having a dilution ratio of 1/10., 1/100, 1/1,000 and 1/10,000, respectively, by mixing 1 ml of the original sample with 9 ml of water to form a 1/10 dilution, mixing 1 ml of the 1/10 dilution with 9 ml of water to form a 1/100 dilution, etc.

In addition, 3 dilution samples were prepared for the yeast sample, having dilution ratios of 1/10, 1/100 and 1/1000, and 2 dilution samples were prepared for the mold sample, having dilution ratios of 1/10 and 1/100. Thus, a total of 48 test samples were prepared from the original 12 test samples, i.e., the original test bacteria test sample plus 4 dilutions thereof, the original yeast test sample plus 3 dilutions thereof, and the original mold test sample plus 2 dilutions thereof.

Preparation of diluted test samples was undertaken as a cautionary step, in that formation of colonies with certain dilutions of original test sample may be uncountable. By having various dilutions of test samples to examine, it was possible to count the colonies of microbial growth in a sample, regardless of the rate of production thereof.

100 μl of each of the test samples was then extracted and injected into an agar medium using a culture medium rod, to provide 5, 4 and 3 agar medium test samples, respectively, of each of the 12 original samples 1-12 (i.e., 48 agar medium test samples total were prepared at the 2 week mark, respectively). The samples containing mold or yeast were spread on an agar medium formed of potato dextrose, whereas the samples containing bacteria were spread on an agar medium formed of nutrient agar.

These 48 agar medium test samples were then placed in a constant temperature environment for 2-3 days, at which time the colony growth was countable. The agar medium test samples containing mold or yeast were maintained at 25° C., whereas the agar medium test samples containing bacteria were maintained at 30° C.

The microbial content of each of the compositions was then determined by visually counting the number of colonies growing on the agar medium test samples. In instances in which the colony growth was excessive in the original undiluted sample (i.e., where counting of individual colonies was impossible due to excessive growth thereof), the colony growth was calculated by calculation of the actual number based on the dilution rate and colony number counted.

Further, agar medium test samples were prepared as described above at additional intervals of 3 and 4 weeks. The number of colonies in these diluted samples was again counted as described above.

Finally, the effectiveness of the test compositions were determined based on the rating system below, and the results of said tests shown in Table I below:

For bacteria, a 99.9% or more decrease in microbial concentration was observed within 2 weeks, for yeast, a 99% or more decrease was observed, and for mold, a 90%

or more decrease was observed=

[symbol]

Same parameters as above within 3 weeks=⋄

Same parameters as above with in 4 weeks=Δ

For bacteria, concentration decreased less than 99.9%, for yeast, less than 99% decrease was observed, and for mold, a less than 90% decrease was observed, within 4 weeks=X

TABLE 4-I

| | | 3-Methyl- | | | | | |
|---|---|---|---|---|---|---|---|
| | 1,2-Pentanediol | 3-Methoxybutanol | Mold | Yeast | Bacteria | | Total |
| Sample # | (wt %) | (wt %) | (cfu/ml) | (cfu/ml) | (cfu/ml) | Result | Result |
| 1 | 3 | 0 | 10⁴ | 0 | 0 | X | X |
| 2 | 3 | 0 | 0 | 10⁵ | 0 | Δ | |
| 3 | 3 | 0 | 0 | 0 | 10⁶ | □ | |
| 4 | 3 | 1 | 10⁴ | 0 | 0 | ⋄ | ⋄ |
| 5 | 3 | 1 | 0 | 10⁵ | 0 | [symbol] | |
| 6 | 3 | 1 | 0 | 0 | 10⁶ | □ | |
| 7 | 3 | 3 | 10⁴ | 0 | 0 | [symbol] | [symbol] |
| 8 | 3 | 3 | 0 | 10⁵ | 0 | [symbol] | |
| 9 | 3 | 3 | 0 | 0 | 10⁶ | [symbol] | |
| 10 | 0 | 3 | 10⁴ | 0 | 0 | X | X |
| 11 | 0 | 3 | 0 | 10⁵ | 0 | X | |
| 12 | 0 | 3 | 0 | 0 | 10⁶ | ⋄ | |

The results shown above demonstrate that 1,2-pentanediol and 3-methyl-3-methoxybutanol when used in combination in a topical solution, provide unexpectedly superior antibacterial preservative effects in the cosmetic. In particular, the results of the above tests illustrate the unexpectedly superior antibacterial/antifungal effects obtained with the instant invention, wherein no paraben is needed to obtain such antimicrobial/preservative effects.

1. A method of preserving a cosmetic comprising mixing therein a substantially paraben-free endermic liniment composition comprising an antiseptic/antifungal agent for inhibition of microbial growth in cosmetics, said agent comprising:
0.1-3 wt % 2-n-butyl-2-ethyl-1,3-propanediol,
2.0-5.0 wt % 1,3-butylene glycol, and water.

2. The method of claim 1, wherein said composition is a moisturizing cream comprising 40-60 wt % water.

3. A method of preserving a cosmetic comprising mixing therein an antiseptic/antifungal agent, said agent comprising:
(a) 0.1-3.0 wt % 2,2-dialkyl-1,3-propanediol; and
(b) 0.1-15.0 wt % of a diol selected from the group consisting of propylene glycol, 1,3-butylene glycol, 1,2-pentanediol, dipropylene glycol, 1,2-butylene glycol, 2,5-pentanediol, 2,4-pentanediol, 2-methyl-2,5-pentanediol, 1,2-hexylene glycol, and 1,6-hexylene glycol.
